(19) United States
(12) Patent Application Publication
(10) Pub. No.: US 2005/0014846 A1
(43) Pub. Date: Jan. 20, 2005

(54) Ambroxol for the treatment of tinnitus

(75) Inventors: Manfred Pilgramm, Detmold (DE); Thomas Weiser, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/888,371

(22) Filed: Jul. 9, 2004

(30) Foreign Application Priority Data
Jul. 16, 2003 (DE) DE 103 32 472

(51) Int. Cl. A61K 31/137

(52) U.S. Cl. 514/650

(57) ABSTRACT

A method of treating an otological disorder in a patient in need thereof, the method comprising administering to the patient ambroxol or a pharmacologically acceptable salt thereof.

### RELATED APPLICATIONS

This application claims priority to German patent application No. DE 103 32 472.0, filed Jul. 16, 2003, which is hereby incorporated by reference in its entirety.

### FIELD OF THE INVENTION

The invention relates to the use of ambroxol and the pharmacologically acceptable salts thereof for preparing a pharmaceutical composition for the treatment of tinnitus and other otological disorders, for example, auditory hyperesthesia, Menière's disease, or endolymphatic hydrops.

### BACKGROUND OF THE INVENTION

The active substance ambroxol (trans-4-(2-amino-3,5-dibromobenzylamino)cyclohexanol) is a known local anesthetic, antitussive, and expectorant. In addition, ambroxol's effect as a sodium channel blocker is described in the literature (Society for Neuroscience Abstracts, 2000, Vol. 26, No. 1-2). Sodium channel blockers are primarily known as local anesthetics. It is known from the prior art that individual sodium channel blockers may have a positive effect on tinnitus (such as, e.g., lidocaine, Hartigh et al., 1993, Clin. Pharm. and Ther. 54, 415-420).

In addition to sodium channel blockers, calcium channel blockers have also been discussed for the treatment of tinnitus (Davies, Knox and Donaldson, British Journal of Audology 28: 125ff, 1994; Shulman, International Tinnitus Journal 3: 77ff, 1997). Antagonists of ionotropic glutamate receptors, particularly of AMPA (α-amino-3-hydroxy-5-methyl-4-isoxazolepropionate) receptors, have been particularly recommended for the treatment of tinnitus (Shulman, International Tinnitus Journal 3: 77-93, 1997), but have not yet achieved recognition for this indication. The taking of hitherto known sodium channel blockers for the treatment of tinnitus is often bound up with central nervous and cardiovascular side-effects.

Oral administration of the medicament, which would be advantageous for the patient, is not possible in the case of lidocaine, for example, which has to be administered intravenously.

The aim of the present invention is therefore to provide an active substance for the treatment of otological disorders, for example, tinnitus, auditory hyperesthesia, Menière's disease, or endolymphatic hydrops, particularly tinnitus, which has no or only negligible central nervous and cardiovascular side-effects. In addition, the active substance thus prepared should be suitable for oral administration.

### DESCRIPTION OF THE INVENTION

Surprisingly, ambroxol exhibits very good activity in the treatment of otological disorders, particularly tinnitus, this activity being based among other things on the blockade of over-activated voltage-dependent sodium channels. At a pharmaceutically effective dose, there are no central nervous and cardiovascular side-effects. Surprisingly ambroxol also exhibits very good effects as a calcium channel blocker and as an AMPA receptor antagonist, which complement the treatment of tinnitus in an ideal manner.

The invention therefore relates to the use of ambroxol or one of the pharmacologically acceptable salts thereof for preparing a pharmaceutical composition for the systemic treatment of otological disorders, preferably tinnitus, auditory hyperesthesia, Menière's disease, or endolymphatic hydrops, most preferably tinnitus.

Preferably, ambroxol or one of the pharmacologically acceptable salts thereof is used to prepare a pharmaceutical composition for the treatment of tinnitus.

The invention further relates to the use of an orally administered pharmaceutical composition, preferably in the form of a tablet, containing ambroxol or one of the pharmacologically acceptable salts thereof.

It is also preferred to use ambroxol or one of the pharmacologically acceptable salts thereof as described above, the ambroxol being used in a daily dosage of 30 mg to 4000 mg, preferably 150 mg to 3000 mg, more preferably 350 mg to 2500 mg, and most preferably 500 mg to 2000 mg.

It is particularly preferred to use ambroxol or one of the pharmacologically acceptable salts thereof for preparing a pharmaceutical composition for the treatment of auditory hyperesthesia, Menière's disease, or endolymphatic hydrops.

The invention further relates to a pharmaceutical composition containing ambroxol and one or more active substances selected from among the calcium channel antagonists, glutamate receptor antagonists, particularly glutamate receptor antagonists of the NMDA or AMPA subtype, CGRP agonists, CGRP antagonists, anticonvulsants of the baclofen type, and osmoregulators, for example, mannitol, glycerol, and frusemide.

The invention also relates to the use of ambroxol or one of the pharmacologically acceptable salts thereof in combination with one or more other active substances selected from the group consisting of analgesics, antidepressants, calcium channel antagonists, glutamate receptor antagonists, particularly glutamate receptor antagonists of the NMDA or AMPA subtype, CGRP agonists, CGRP antagonists, anticonvulsants of the baclofen type, osmoregulators, for example, mannitol, glycerol, or frusemide, sodium channel blockers, anticonvulsants, for example, barbiturates or benzodiazepines, antiarrhythmics, and neuroprotectives.

Preferably, ambroxol or one of the pharmacologically acceptable salts thereof is used in combination with one or more analgesics selected from the group consisting of opioids, non-steroidal analgesics, gabapentin, and alpha-adrenergic agonists.

The name ambroxol within the scope of the present invention denotes both the base ambroxol and also the solvates or hydrates thereof, preferably the base ambroxol.

Acids suitable for forming salts of ambroxol are, for example, hydrochloric acid, hydrobromic acid, sulfuric acid, phosphoric acid, nitric acid, oxalic acid, malonic acid, fumaric acid, maleic acid, tartaric acid, citric acid, ascorbic acid, and methanesulfonic acid, preferably hydrochloric acid.

The effects of ambroxol according to the invention will be illustrated by the Examples that follow. These serve merely to illustrate the invention and are not to be regarded as limiting it.

Ambroxol inhibits various neuronal sodium channel subtypes, the half-maximum inhibition of centrally expressed channels being 111 μM (Weiser and Wilson, Mol. Pharmacol. 62, 433-438, 2002).

Voltage-dependent calcium channels play an important role in neurotransmission. It has been found, surprisingly, that ambroxol also blocks voltage-dependent calcium channels in neuron cultures from rats in concentrations of 10 μM to 1000 μM. Neurons were dissected from posterior root ganglia of adult rats and placed in short-term culture. The cells were investigated electrophysiologically by the Patch-Clamp method (voltage terminal), and the flow of current through voltage-dependent calcium channels was measured after electrical stimulation (voltage jumps from −80 mV to 0 mV holding potential for 50 ms) in the presence and absence of ambroxol.

Ionotropic glutamate receptors of the AMPA subtype are also essential for the excitatory neurotransmission. In HEK 293 cells which express heterologously human GluR1/2 receptors, ambroxol surprisingly inhibits glutamate-induced membrane currents in concentrations ranging from 30-1000 μM. HEK 293 cells which expressed functionally recombinant human GluR1/2 receptors were electrophysiologically investigated by the Patch-Clamp method (voltage terminal). The administration of 1 mM glutamate (for 1 s at a holding potential of −80 mV) induced membrane currents which were inhibited by the joint administration of ambroxol.

Ambroxol may be used on its own or in combination with other pharmacologically active substances. Suitable preparations include, for example, tablets, capsules, suppositories, solutions, elixirs, emulsions, or dispersible powders. Suitable tablets may be obtained, for example, by mixing the active substance(s) with known excipients, for example, inert diluents such as calcium carbonate, calcium phosphate, or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example, collidone or shellac, gum arabic, talc, titanium dioxide, or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol, or sugar and a flavor enhancer, e.g., a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g., with the addition of preservatives such as p-hydroxybenzoates, or stabilizers such as alkali metal salts of ethylenediamine tetraacetic acid, and transferred into injection vials or ampoules.

Capsules containing one or more active substances or combinations of active substances may, for example, be prepared by mixing the active substances with inert carriers such as lactose or sorbitol and packing them into gelatine capsules.

Suitable suppositories may be made, for example, by mixing with carriers provided for this purpose, such as neutral fats or polyethyleneglycol or the derivatives thereof.

A therapeutically effective daily dose is between 30 mg to 4000 mg, preferably 150 mg to 3000 mg, more preferably 350 mg to 2500 mg, most preferably 500 mg to 2000 mg of ambroxol in adults.

The Examples which follow illustrate the present invention without restricting its scope:

Examples of Pharmaceutical Formulations

A)

| Tablets | per tablet (mg) |
|---|---|
| ambroxol | 800 |
| lactose | 140 |
| maize starch | 240 |
| polyvinylpyrrolidone | 20 |
| magnesium stearate | 10 |

Ambroxol, lactose and some of the maize starch are mixed together. The mixture is screened, then moistened with a solution of polyvinylpyrrolidone in water, kneaded, wet-granulated, and dried. The granules, the remaining maize starch, and the magnesium stearate are screened and mixed together. The mixture is compressed to produce tablets of suitable shape and size.

B)

| Tablets | per tablet (mg) |
|---|---|
| ambroxol | 800 |
| maize starch | 190 |
| lactose | 55 |
| microcrystalline cellulose | 35 |
| polyvinylpyrrolidone | 20 |
| sodium-carboxymethyl starch | 30 |
| magnesium stearate | 10 |

Ambroxol, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

C)

| Coated tablets | per coated tablet (mg) |
|---|---|
| ambroxol | 500 |
| maize starch | 45 |
| lactose | 30 |
| polyvinylpyrrolidone | 5 |
| magnesium stearate | 5 |

Ambroxol, maize starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 11 mm are compressed in a tablet-making machine. The tablet cores thus produced are coated in known manner with a covering consisting essentially of sugar and talc. The finished coated tablets are polished with wax.

D)

| Capsules | per capsule (mg) |
|---|---|
| Ambroxol | 250 |
| maize starch | 268.5 |
| magnesium stearate | 1.5 |

Ambroxol and maize starch are mixed and moistened with water. The moist mass is screened and dried. The dry granules are screened and mixed with magnesium stearate. The finished mixture is packed into size 1 hard gelatine capsules.

E)

| Parenteral solution | |
|---|---|
| Ambroxol | 500 mg |
| Citric acid monohydrate | 100 mg |
| sodium hydroxide | 35 mg |
| mannitol | 1500 mg |
| water for inj. | 50 mL |

The ambroxol is dissolved in water at its own pH or optionally at pH 5.5 to 6.5 and mannitol is added to make it isotonic. The solution obtained is filtered free from pyrogens and the filtrate is transferred under aseptic conditions into injection vials which are then sealed with rubber stoppers and autoclaved.

F)

| Suppositories | |
|---|---|
| Ambroxol | 450 mg |
| solid fat | 1650 mg |

The hard fat is melted. At 40° C. ambroxol is homogeneously dispersed therein. The mixture is cooled to 38° C. and poured into slightly chilled suppository moulds.

G)

| Oral solution | | |
|---|---|---|
| ambroxol | 150 | mg |
| hydroxyethylcellulose | 50 | mg |
| sorbic acid | 5 | mg |
| sorbitol (70%) | 600 | mg |
| glycerol | 200 | mg |
| flavoring | 15 | mg |
| water | to 10 | mL |

Distilled water is heated to 70° C. Hydroxyethylcellulose is dissolved therein with stirring. After the addition of sorbitol solution and glycerol, the mixture is cooled to ambient temperature. At ambient temperature sorbic acid, flavoring, and ambroxol are added. To eliminate air from the suspension, it is evacuated with stirring.

H)

| Ointment | | |
|---|---|---|
| Composition g/100 g ointment | | |
| ambroxol | 20 | g |
| sodium disulfite | 0.1 | g |
| acetyl alcohol | 10 | g |
| stearyl alcohol | 10 | g |
| white vaseline | 5 | g |
| perfume oil | q.s. | |
| distilled water | to 100 | g |

The ingredients are processed in the usual way to form an ointment.

1. A method of treating an otological disorder in a patient in need thereof, the method comprising administering to the patient ambroxol or a pharmacologically acceptable salt thereof.

2. The method according to claim 1, wherein the otological disorder is tinnitus.

3. The method according to claim 1, wherein the otological disorder is auditory hyperesthesia, Menière's disease, or endolymphatic hydrops.

4. The method according to claim 1, wherein the ambroxol or a pharmacologically acceptable salt thereof is administered orally to the patient.

5. The method according to one of claims 1 to 4, wherein the ambroxol or a pharmacologically acceptable salt thereof is administered to the patient in a daily dose of 30 mg to 4000 mg.

6. The method according to one of claims 1 to 4, wherein one or more additional active substances selected from calcium channel antagonists, glutamate receptor antagonists, CGRP agonists, CGRP antagonists, anticonvulsants of the baclofen type, and osmoregulators are administered to the patient before, after, or simultaneously with the ambroxol or a pharmacologically acceptable salt thereof.

7. The method according to one of claims 1 to 4, wherein one or more additional active substances selected from analgesics, antidepressants, calcium channel antagonists, glutamate receptor antagonists, CGRP agonists, CGRP antagonists, anticonvulsants of the baclofen type, osmoregulators, sodium channel blockers, anticonvulsants, antiarrhythmics, and neuroprotectives are administered to the patient before, after, or simultaneously with the ambroxol or a pharmacologically acceptable salt thereof.

8. The method according to one of claims 1 to 4, wherein one or more additional active substances selected from analgesics selected from the group consisting of opioids, non-steroidal analgesics, gabapentin, and alpha-adrenergic agonists are administered to the patient before, after, or simultaneously with the ambroxol or a pharmacologically acceptable salt thereof.

9. A pharmaceutical composition comprising:
(a) ambroxol or a pharmacologically acceptable salt thereof; and
(b) one or more additional active substances selected from calcium channel antagonists, glutamate receptor antagonists, CGRP agonists, CGRP antagonists, anticonvulsants of the baclofen type, and osmoregulators.

10. A pharmaceutical composition comprising:
(a) ambroxol or a pharmacologically acceptable salt thereof; and
(b) one or more additional active substances selected from analgesics, antidepressants, calcium channel antagonists, glutamate receptor antagonists, CGRP agonists, CGRP antagonists, anticonvulsants of the baclofen type, osmoregulators, sodium channel blockers, anticonvulsants, antiarrhythmics, and neuroprotectives.

11. A pharmaceutical composition comprising:
(a) ambroxol or a pharmacologically acceptable salt thereof; and
(b) one or more analgesics selected from the group consisting of opioids, non-steroidal analgesics, gabapentin, and alpha-adrenergic agonists.
